(19) United States
(12) Patent Application Publication
(10) Pub. No.: US 2018/0020967 A1
(43) Pub. Date: Jan. 25, 2018

(54) SWEAT SENSING DEVICES WITH EXCESS SWEAT FLOW MANAGEMENT

(71) Applicant: Eccrine Systems, Inc., Cincinnati, OH (US)

(72) Inventors: Robert Beech, Cincinnati, OH (US); Jason Heikenfeld, Cincinnati, OH (US); Gavi Begtrup, Cincinnati, OH (US); Austin Morgan, Cincinnati, OH (US); James Bostik, Cincinnati, OH (US)

(73) Assignee: Eccrine Systems, Inc., Cincinnati, OH (US)

(21) Appl. No.: 15/657,206

(22) Filed: Jul. 24, 2017

Related U.S. Application Data
(60) Provisional application No. 62/366,107, filed on Jul. 24, 2016.

(51) Int. Cl. A61B 5/00; A61B 5/145

(52) U.S. Cl. CPC A61B 5/4266; A61B 5/6801; A61B 5/14517

(57) ABSTRACT

A wearable sweat sensing device is provided for managing excess sweat flow. The sweat sensing device includes at least one sweat sensor and a sweat path for conveying sweat from a wearer's skin and across the at least one sensor. At least one adaptive sweat flow component is in fluidic communication with the sweat path for removing excess sweat flow from the sweat path. The adaptive sweat flow component can include one or more of an adaptive sweat sample collector, an adaptive sweat sample channel, or an adaptive sweat sample pump. A method of using the disclosed device to remove excess sweat flow from the device's sweat path during periods of high sweat rate is also provided.

### CROSS-REFERENCE TO RELATED APPLICATIONS

The present application claims priority to U.S. Provisional Application No. 62/366,107, filed Jul. 24, 2016, and has specification that relates to PCT/US13/35092, the disclosures of which are hereby incorporated by reference herein in their entirety.

### BACKGROUND OF THE INVENTION

Sweat sensing technologies have enormous potential for applications ranging from athletics, to neonatology, to pharmacological monitoring, to personal digital health, to name a few applications. Sweat contains many of the same biomarkers, chemicals, or solutes that are carried in blood and can provide significant information enabling one to diagnose illness, health status, exposure to toxins, performance, and other physiological attributes even in advance of any physical sign. Furthermore, sweat itself, the action of sweating, and other parameters, attributes, solutes, or features on, near, or beneath the skin can be measured to further reveal physiological information.

Despite the significant potential of sweat as a sensing paradigm, technology has not advanced beyond decades-old usage in infant chloride assays for Cystic Fibrosis or illicit drug monitoring patches. Currently, the majority of research efforts on sweat sensing technology utilize the slow and inconvenient process of sweat stimulation, collection of a sample, transport of the sample to a lab, and then analysis of the sample by a bench-top machine and a trained expert. This process is so labor intensive, complicated, and costly that in most cases a blood test would be a superior modality, since it is the gold standard for most forms of high performance biomarker sensing. Hence, sweat sensing has not emerged into its fullest opportunity and capability for biosensing, especially for continuous or repeated biosensing or monitoring. Furthermore, attempts at using sweat to sense “holy grails” such as glucose have not yet succeeded to produce viable commercial products, reducing the publically perceived capability and opportunity space for sweat sensing.

Of all the other physiological fluids used for bio monitoring (e.g., blood, urine, saliva, tears), sweat has arguably the least predictable sampling rate in the absence of technology. However, with proper application of technology, sweat can be made to outperform other non-invasive or less invasive biofluids in predictable sampling. For example, it is difficult to control saliva or tear rate without negative consequences for the user (e.g., dry eyes, tears, dry mouth, or excessive saliva while talking). Urine is also a difficult fluid for physiological monitoring, because it is inconvenient to take multiple urine samples, it is not always possible to take a urine sample when needed, and control of biomarker dilution in urine imposes further significant inconveniences on the user or test subject.

A number of sweat sensing device applications require operation at very low sweat generation rates, for example, the detection of large proteins and other molecules that diffuse more slowly into sweat. Other applications necessitate minimizing or even eliminating sweat stimulation. However, current device configurations for low sweat generation rates produce a risk of operational failure during temporary or sustained periods of high sweat generation rates. Excessive sweat flow may overwhelm the wicking capacities, osmotic filter capacities, and other components' fluid capacities in the device. Therefore, there is a need for sweat sensing devices that can operate at low sweat generation rates, but are configured to manage periods of excessive sweat flow. Such improvements are the subject of the present disclosure.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sweat sensing technology into intimate proximity with sweat as it is generated. With such an invention, sweat sensing could become a compelling new paradigm as a biosensing platform.

### SUMMARY OF THE INVENTION

Wearable sweat sensing devices are described herein which can detect analytes in sweat at very low sweat generation rates, while also managing temporary or sustained periods of very high sweat generation rates. In a first aspect, a sweat sensing device is provided for placement on a wearer's skin. The device includes at least one sweat sensor, and a sweat path, where the sweat path includes: a sweat sample collector, a microfluidic channel, and a pump, and where the path function to convey sweat from the skin and across at least one sweat sensor and away from the sensor. The device also includes at least one adaptive sweat flow component in fluidic communication with the sweat path, where the adaptive component is configured to remove excess sweat from the sweat path. In a second aspect, a method of managing excessive sweat flow in a wearable sweat sensing device is provided. The method includes drawing sweat flow from a wearer's skin into the device and conveying the sweat flow through a sweat path containing at least one sensor for measuring sweat. The method further includes drawing excess sweat away from the sweat path when the generated sweat rate exceeds the capacity of the device.

### BRIEF DESCRIPTION OF THE DRAWINGS

The disclosed invention will be further appreciated in light of the following descriptions and drawings in which:

FIG. 1A is a diagrammatic view of a wearable device for sweat biosensing;

FIG. 1B is a cross-sectional view of at least a portion of a wearable device for sweat biosensing;

FIG. 2A is a side, cross-sectional view of a first exemplary embodiment of a wearable device for sweat biosensing with adaptive sweat flow components;

FIG. 2B is a top view of the biosensing device shown in FIG. 2A;

FIG. 2C is a cross-sectional view of the biosensing device of FIG. 2B taken along line 2C-2C;

FIG. 2D is a perspective view of the biosensing device of FIG. 2A;

FIG. 3A is a side, cross-sectional view of a second exemplary embodiment of a wearable sweat biosensing device;

FIG. 3B is a side, cross-sectional view of the device of FIG. 3A, showing a primary adaptive material swelled into contact with a secondary adaptive material;

FIG. 4A is a top, schematic view of another exemplary embodiment for a wearable sweat biosensing device having at least one adaptive sweat flow component;

FIG. 4B is a top, schematic view of the biosensing device of FIG. 4A, showing an adaptive component in a sweat release position;

FIG. 5 is a top, schematic view showing at least a portion of another exemplary embodiment for a wearable sweat biosensing device with adaptive components; and

FIG. 6 is a top, schematic view showing at least a portion of another exemplary embodiment of a wearable sweat biosensing device with adaptive components.

### DEFINITIONS

“Fluid resistance” means the relative speed and volume with which a fluid can wick through a material. High fluid resistance materials wick and absorb sweat slowly.

“Chronological assurance” means a sampling rate or sampling interval for measurement(s) of sweat, or solutes in sweat, at which measurements can be made of new sweat or its new solutes as they originate from the body. Chronological assurance may also include a determination of the effect of sensor function, or potential contamination with previously generated sweat, previously generated solutes, other fluid, or other measurement contamination sources for the measurement(s).

“Sweat sampling rate” means the effective rate at which new sweat, or sweat solutes, originating from the sweat gland or from skin or tissue, reaches a sensor that measures a property of sweat or its solutes. Sweat sampling rate, in some cases, can be far more complex than just sweat generation rate. Sweat sampling rate directly determines, or is a contributing factor in determining chronological assurance. Times and rates are inversely proportional (rates having at least partial units of 1/seconds), therefore a short or small time required to refill a sweat volume can also be said to have a fast or high sweat sampling rate. The inverse of sweat sampling rate (1/s) could also be interpreted as a “sweat sampling interval(s)”. Sweat sampling rates or intervals are not necessarily regular, discrete, periodic, discontinuous, or subject to other limitations. Like chronological assurance, sweat sampling rate may also include a determination of the effect of potential contamination with previously generated sweat, previously generated solutes, other fluid, or other measurement contamination sources for the measurement(s). Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources. Sensor response times may also affect sampling rate.

“Sweat generation rate” means the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

“Measured” may mean an exact or precise quantitative measurement and can include broader meanings such as, for example, measuring a relative amount of change of something. Measured can also mean a binary measurement, such as ‘yes’ or ‘no’ type qualitative measurements.

“Analyte-specific sensor” means a sensor that is specific to an analyte in sweat. Simply measuring sweat conductivity is not specific to one analyte because it measures the sum of conductance contributed by all ionic solutes in sweat. However, an ion-selective electrode configured to detect potassium is a sensor specific to one analyte. As an additional example, a sensor for sweat cortisol that only has interference (non-specificity) to estrogen, would still be specific to one analyte as described herein, since there are many device applications in which estrogen concentrations are static, but cortisol concentrations would change, making the sensor effectively specific to cortisol.

“Sweat volume” means the fluidic volume in a space that can be defined multiple ways. Sweat volume may be the volume that exists between a sensor and the point of generation of sweat, or between a sensor and a solute moving into or out of sweat from the body or from other sources. Sweat volume can include the volume that can be occupied by sweat between the sampling site on the skin and a sensor on the skin, where the sensor has no intervening layers, materials, or components between it and the skin; or between the sampling site on the skin and a sensor on the skin where there are one or more layers, materials, or components between the sensor and the sampling site on the skin. Sweat volume may refer to the sweat volume of multiple integrated components, or used in description of the sweat volume for single component or a subcomponent, or in the space between a device, or device component, and skin.

“Microfluidic components” means channels in polymer, textiles, paper, or other components known in the art of microfluidics for guiding movement of a fluid or at least partial containment of a fluid.

“Wicking pressure,” “wicking force,” “capillary pressure,” or “capillary force,” means a pressure or force that should be interpreted according to its general scientific meaning. For example, a capillary (tube) geometry can be said to have a capillary pressure or a wicking pressure. Or a wicking textile or gel may have a capillary pressure, even if the material is not geometrically a tube or a channel. Conversely, a wicking fiber can have an effective capillary pressure. Similarly, the (relatively empty) space between a material placed on skin and the skin surface can have an effective wicking pressure. The terms wicking or capillary pressure and wicking or capillary force may be used interchangeably herein to describe the effective pressure provided by any component or material that is capable of capturing sweat by a negative pressure (i.e., pulling it into or along said component or material). For simplicity, the term “wicking pressure” will be used herein to refer to any of the above alternate terms. Wicking pressure also must be considered in its specific context, for example, if a sponge is fully saturated with water, then it has no remaining wicking pressure. Wicking pressure must therefore be interpreted as described in the specification for a device during use, and not interpreted in isolation or in contexts other than the disclosed devices or use scenarios.

As used herein, “sweat path” includes the following: a sweat sample collector, a microfluidic channel, and a pump, and where the path functions to convey sweat from the skin and across at least one sweat sensor and away from the sensor.

“Sweat sample collector” means any component of the disclosed invention that supports the creation of, or sustains, a volume reduced pathway, or that is the component that receives sweat before a device sensor and is on or adjacent to skin. A collector can be a microfluidic component, a capillary material, a wrinkled surface, a textile, a gel, a coating, a film, or any other component that satisfies the general criteria of the present disclosure. A collector may be part of the same component or material that serves other purposes (e.g., a pump or a channel), and in such cases, the portion of said component or material that at least in part receives sweat before the sensor(s) and is on or adjacent to skin is also a sweat sample collector as defined herein.

“Sweat sample channel” means any component of the disclosed invention that is on or adjacent to a sweat sensing device sweat sample collector and that promotes transport of sweat or its solutes by wicking pressure, advective flow, diffusion, or other method of transport, from the collector, across device sensors and to a sweat sample pump. In some embodiments, the channel function may be performed by a suitably configured sweat collector. A channel may be part of the same component or material that serves other purposes (e.g., a sweat collector or a sweat sample pump), and in such cases, the portion of said component or material that, at least in part, fluidically connects the collector to the pump and conveys sweat to a sensor(s) and that is on or adjacent to the sensor(s), is also a channel as defined herein.

“Sweat sample pump” refers to any component of the disclosed invention that supports creation of or sustains a volume reduced pathway, or that receives sweat after a sweat sensing device sensor and has a primary purpose of collecting excess sweat to allow sustained operation of the device. A pump may also include an evaporative material or surface that is configured to remove excess sweat by evaporation of water. A pump may be part of the same component or material that serves other purposes (e.g., a sweat collector or a sweat channel), and in such cases, the portion of said component or material that at least in part receives sweat after the sensor(s), is also a pump as defined herein.

The term “pump” may also reference alternate configurations, such as a small mechanical pump, or osmotic pressure across a membrane, so long as the pressure generated satisfies the requirements described herein, and the other materials or components between the pump and skin operate by wicking pressure to maintain their respective sweat volumes. For example, a suctioning system that is air-tight to skin would not be considered a sweat sample pump because the disclosed invention permits introduction of air or gas between the device and skin.

“Sweat flow adaptive component” means a component in fluidic communication with a sweat path that is configured to remove excess sweat from the sweat path, where the adaptive component is at least one of a collector, a channel, and a pump.

### DETAILED DESCRIPTION OF THE INVENTION

To provide the proper context for sweat sampling rate and, therefore, chronological assurance, the following description of sweat generation rate and sweat volume is provided. From Dermatology: an illustrated color text, 5th ed., the maximum sweat generated per person per day is 10 L, which on average is 4 μL per gland maximum per day, or about 3 nL/min/gland. This sweat generation rate is about 20× higher than the minimum sweat generation rate. With respect to stimulated sweat generation rates, the maximum sweat generation rate by pilocarpine stimulation is about 4 nL/min/gland for untrained men and 8 nL/min/gland for trained (exercising often) men. Buono et al., Cholinergic sensitivity of the eccrine sweat gland in trained and untrained men, J. Derm. Sci., Vol. 4, Issue 1, 33-37 (1992). Other sources indicate that the maximum sweat generation rates for an adult can be up to 2-4 L per hour or 10-14 liters per day (10-15 g/min·m²), which based on the per hour number translates to a range of 3 nL/min/gland to 20 nL/min/gland. Sweat stimulation data from “Pharmacologic responsiveness of isolated single eccrine sweat glands,” by K. Sato and F. Sato, Am. Physiological Society, Jul. 30, 1980, suggests that a sweat generation rate of up to about 5 nL/min/gland is possible with stimulation, and several types of sweat stimulating substances are disclosed (the data was for extracted and isolated monkey sweat glands, which are very similar to human ones). For simplicity, we can assume for calculations in the present disclosure (without so limiting the disclosure), that the minimum sweat generation rate is about 0.1 nL/min/gland, and the maximum sweat generation rate is about 5 nL/min/gland, which is about a 50× difference between the maximum and minimum rates.

Based on the assumption of a sweat gland density of approximately 100 glands/cm², a sensor that is 0.55 cm in radius (1.1 cm in diameter) would cover an area of about 1 cm², or approximately 100 sweat glands. A skin-facing sensor having that same 1 cm² area and an average height of 100 μm, or 100E-4 cm, would provide a sweat volume (in the space between the sensor and the skin) of 100E-4 cm³ or about 100E-4 mL (10 μL). At the maximum sweat generation rate of 5 nL/min/gland and 100 glands, it would require 20 minutes to fully refresh the sweat volume (using first principles/simplest calculation only). At the minimum sweat generation rate of 0.1 nL/min/gland and 100 glands, it would require 1000 minutes or approximately 17 hours to refresh the sweat volume. If the flow is not fully centered on the sensor, according to Sonner, et al., in Biomicrofluidics, 2015 May 15; 9(3):031301. doi: 10.1063/1.4921039, the time to fully refresh the sweat volume (e.g., new sweat replaces all old sweat) could be 6× longer or more. Additionally, slow sweat flow rates, back-diffusion of analytes, and other confounding factors could make the effective sampling interval even larger. Clearly, conventional wearable sweat sensing approaches with large sweat volumes and slow sampling rates would find continuous sweat sample monitoring to be a significant challenge.

Sweat stimulation, or sweat activation, can be achieved by known methods. For example, sweat stimulation can be achieved by simple thermal stimulation, a chemical heating pad, infrared light, oral administration of a drug, intradermal injection of drugs such as carbachol, methylcholine or pilocarpine, dermal introduction of such drugs using iontophoresis, sudo-motor-axon reflex sweating, or by other means. A device for iontophoresis may, for example, provide direct current and use large lead electrodes lined with porous material, where the positive pole is dampened with 2% pilocarpine hydrochloride and the negative one with 0.9% NaCl solution. Sweat can also be controlled or created by asking the device wearer to conduct or increase activities or conditions that cause them to sweat.

The present disclosure applies to any type of sweat sensing device that stimulates sweat, or that measures sweat, sweat solutes, and/or solutes that transfer into sweat from skin. Any suitable sensor may be used in the disclosed embodiments, including, for example, ion-selective, enzymatic, antibody, aptamer, optical, electrical, mechanical, and similar types of sensing devices. The disclosure applies to sweat sensing devices with various configurations including patches, bands, straps, portions of clothing, wearables, or any suitable mechanism that reliably brings sweat stimulating, sweat collecting, and/or sweat sensing technology into intimate proximity with sweat as it is generated. Some embodiments use adhesives to hold the device near the skin, but devices may also be secured by other suitable mechanisms including, for example, a strap or helmet suspension.

Certain embodiments of the disclosure describe sensors as simple individual elements. However, many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a sweat sensor; an impedance sensor; a conductivity sensor; a sweat volume sensor; a sweat generation rate sensor; or a solute generation rate sensor. Certain embodiments show sub-components that may require additional obvious sub-components for use of the device in various applications (such as a battery), which for purposes of brevity and focus on inventive aspects, are not explicitly shown in the diagrams or described in the embodiments. Additionally, embodiments described herein may benefit from mechanical or other means to keep the devices or sub-components firmly affixed to skin, or to provide pressure for facilitating constant contact with skin, or conformal contact with ridges or grooves in skin, as are known to those skilled in the art of wearable devices, patches, bandages, or other technologies or materials that are affixed to skin. Such means are included within the spirit of the disclosed invention.

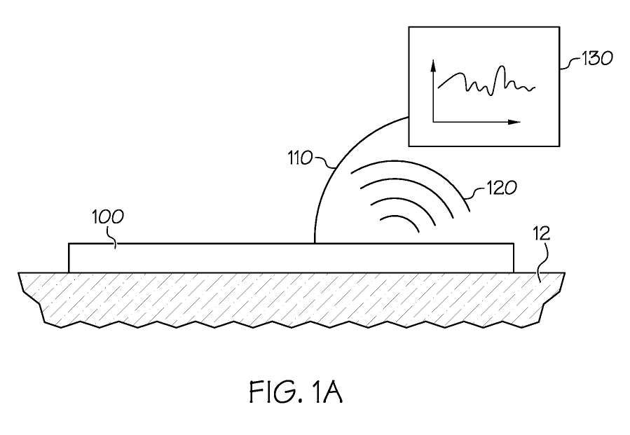

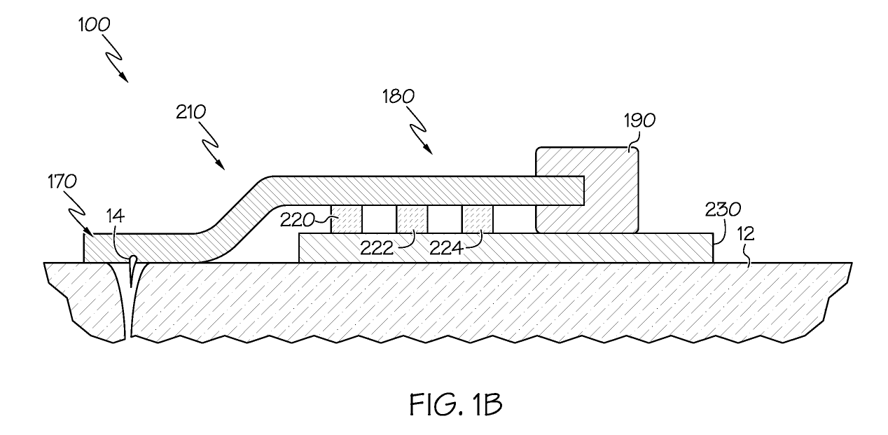

Referring now to the drawing figures, in which like numerals indicate like elements throughout the view, FIG. 1A depicts a wearable sweat sensing device 100 placed on or near skin 12. In alternate embodiments, the sweat sensing device may be fluidically connected to skin or regions near skin by contact with microfluidic components or other suitable techniques. The device 100 is in wired communication 110 or wireless communication 120 with a reader device 130. In some embodiments, the reader device 130 may be a smart phone or portable electronic device. In alternate embodiments, the device 100 and reader device 130 can be combined. In further alternative embodiments, communications 110 and/or 120 may not be constant, but rather a one-time data transmission from the device following sweat sensing.

As shown in greater detail in FIG. 1B, sweat sensing device 100 includes a sweat path 210 for drawing sweat 14 from the skin 12 and conveying the sweat to an analyte-specific primary sensor 220 that is placed on a water impermeable substrate 230. The sweat is conveyed along the path 210 into and from a sweat sample collector 170 and through a channel 180. The primary sensor 220 measures the presence, concentration, or other property of one or more solutes in sweat. For example, the primary sensor 220 can be an impedance sensor for a cytokine biomarker, an ion-selective electrode to measure sodium, or an electrochemical aptamer-based (EAB) sensor to measure cortisol. One or more secondary sensors, indicated at 222, 224 may also be included. These additional sensors may include a drift-free reference electrode, a conductivity sensor, a sensor to detect the presence of sweat, such as a galvanic skin response sensor, a sensor to measure the flow rate of sweat, such as a micro-thermal flow rate sensor, or a temperature sensor. The impermeable substrate 230 can be a polyimide film or similar material. The channel 180 is comprised of a microfluidic component which may be, for example, paper, fabric, a polymer microchannel, a tube, a gel, or other means to transport sweat from skin to the one or more sensors. The device 100 is attached to skin 12 by an adhesive (not shown), which may be, for example, a pressure sensitive, liquid, tacky hydrogel, which promotes robust electrical, fluidic, and iontophoretic contact with skin.

For continuous monitoring, the channel 180 conveys sweat past the one or more sensors 220-224 to a sweat sample pump 190 that continuously draws sweat away from skin 12. The pump 190 draws sweat across sensors 220-224, and subsequently absorbs the sweat, at a rate at which the sweat is typically generated by the skin. In low sweat rate sensing applications, the sweat path 210 will be designed to draw and absorb sweat flow at the desired low sweat generation rate. During temporary or sustained periods of heavy or excessive sweat flow, however, a path that is designed to convey sweat at a minimal rate can be overwhelmed, resulting in a sweat back up through the sweat path 210 which can lead to inaccurate measurements or operational failure of the device.

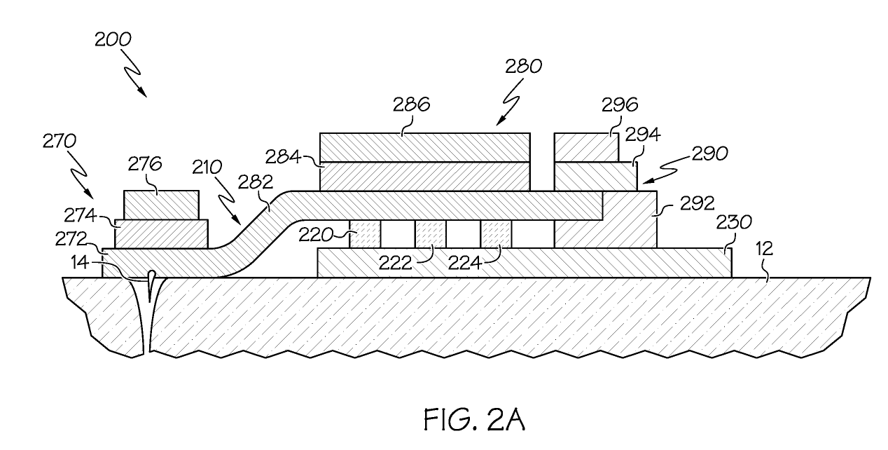

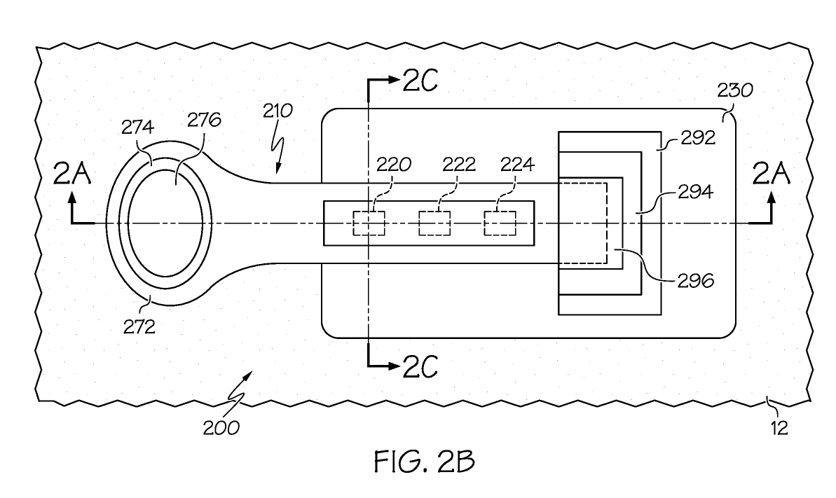

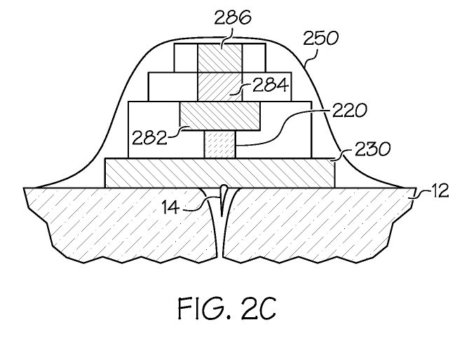

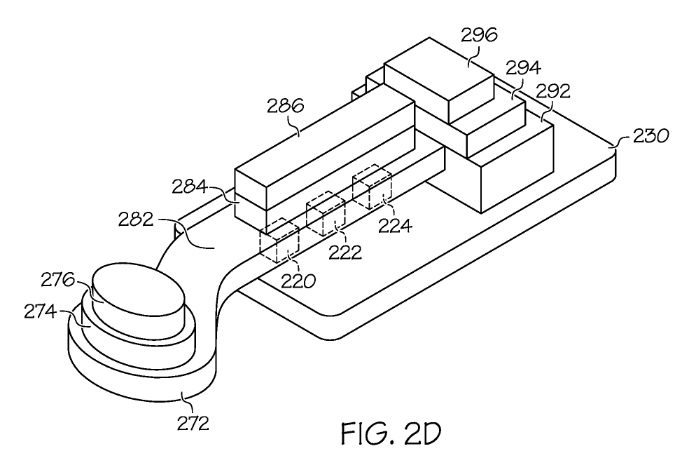

In order to manage excess sweat flow, and thereby minimize sweat accumulation in sweat path 210, sweat sensing devices as disclosed herein include one or more sweat flow adaptive components. In a first exemplary embodiment, depicted in FIGS. 2A-2D, these adaptive components include one or more of an adaptive collector, an adaptive channel, or an adaptive pump for moving excess sweat away from the sweat path 210. The one or more adaptive components facilitate sensing of sweat analytes at very low sweat rates, e.g., 0.1 μL/cm²min-0.5 μL/cm²/min, despite the occurrence of sustained periods of high sweat rates, e.g., 0.9 μL/cm²/min-1.7 μL/cm²/min. As described in more detail below, the sweat flow adaptive components move excess sweat from the sweat path into to fluidically connected materials during periods of high sweat flow, so that a sweat path designed primarily for use with very low sweat rates is not overwhelmed during periods of higher sweat rates.

In the embodiment depicted in FIGS. 2A-2D, the sweat sensing device 200 includes an adaptive collector, indicated at 270, with a primary sweat collecting material 272, having a first fluid resistance, in fluidic communication with skin 12. Primary collecting material 272 forms the sweat collector component of the device's sweat path 210. Primary material 272 is also in fluidic communication with a secondary material 274 having the same or lower fluid resistance than the primary material 272. The fluidic communication between primary material 272 and secondary material 274 can be through physical contact between the layers, or through another type of fluid channel. In some embodiments, the secondary material 274 is, in turn, in fluidic communication with an optional tertiary material 276 having the same or lower fluid resistance than the secondary material. Materials suitable for use as adaptive sweat collector components could include, for example, any combination of cellulose, cellulose acetate, nitrocellulose, or polyester polypropylene. Alternatively, an adaptive collector component may include an array of microchannels embossed into a polymer substrate. The fluid resistance of the component may be altered by varying the material, porosity, hydrophobicity, and/or dimensions of the channels. In some embodiments, the material may have multiple wicking pressures, where the material has a first wicking pressure at one sweat flow rate, and a second, higher wicking pressure at a higher sweat flow rate. For example, Rayon has a first and greater wicking pressure when fluid is wicked along grooves in its fibers, and a second and lower wicking pressure when fluid also fills the spaces in between such fibers.

At low sweat generation rates, sweat will move from the wearer's skin into the primary sweat collecting material 272, and begin to fill and flow through the material. Sweat will move along the path 210 from the collector 270 into the sweat sample channel 280, across the at least one sensor 220 at a sweat sampling rate and into the pump 290. As the sweat generation rate increases, the sweat will continue to be drawn into the device and conveyed across sensor 220 via path 210 up to the path's maximum capacity, but the excess sweat created by the difference in the sweat generation rate and path's capacity will accumulate within the primary sweat collecting material 272. As the primary collecting material 272 reaches saturation, sweat will be wicked from the primary material to the secondary material 274 and begin to fill the secondary material. Moving sweat from the primary material 272 to the secondary material 274 allows the flow rate through the path to be maintained at or near a desired sweat sampling rate. New sweat entering the collector 270 will be moved into the channel 280 and displace the previously collected sweat into the secondary material 274. As the sweat rate increases further, or the sweat flow continues at a rate in excess of the sweat path capacity, the additional excess sweat will saturate the secondary material 274. In some embodiments, as the secondary material 274 fills, sweat will wick from the secondary material to tertiary material 276 and begin to fill the tertiary material. The movement of excessive sweat between the adaptive collector layers 272, 274, 276 enables device 200 to accommodate the excess generated sweat and maintain a chronologically assured sweat sampling rate, without exceeding the capacity of the sweat path 210. In some embodiments, a fluid and vapor resistant cover 250 surrounds the operative components to keep sweat from evaporating out of the device.

As also shown in FIGS. 2A-2D, the sweat sensing device 200 can include an adaptive channel, indicated at 280. The adaptive channel 280 includes a primary channel material 282 that is in fluidic communication with the primary collecting material 272 and the pump 290 for conveying sweat from the collector, across the at least one sensor 220, and to the pump. The primary channel material 282 has a first fluidic resistance, and is also in fluidic communication with a secondary channel material 284 having the same or lower fluid resistance than the primary material. The secondary channel material 284 may, in turn, be in fluidic communication with an optional tertiary channel material 286, where the tertiary material has the same or lower fluid resistance than the secondary material. The adaptive channel components may be stacked as shown, or placed in a side-by-side configuration.

At lower sweat rates, sweat will flow through primary channel material 282 and across sensor 220 at a sweat sampling rate. The sweat flow will at least partially fill the primary material 282 without saturating the material to its outer edges. During periods of excessive sweat flow, sweat will continue to flow across sensor 220 at or near the desired sweat sampling rate. If the higher sweat rate continues, the increasing sweat flow will saturate the primary channel material 282, which in turn will cause sweat to be wicked from the primary material into the secondary channel material 284. The movement of sweat to the secondary material 284 will enable the sweat flow across the sensor 220 to be maintained at or near the desired sweat sampling rate. As sweat rate continues to increase, and the secondary material 284 saturates, excess sweat will continue to expand into the tertiary channel material 286.

The sweat sensing device 200 may also include an adaptive sweat sample pump 290. As with the collector 270 and channel 280, the adaptive pump 290 includes a primary pump material 292, having a first fluid resistance, which is in fluidic communication with the primary channel material 282. The primary pump material 292 at least partially draws a sweat sample from the collector 270, through the primary channel material 282, and across and away from the sensor 220. During periods of excessive sweating, the sweat flowing through the sweat path 210 may overwhelm the capacity of the primary material 292. To accommodate the excess sweat, the primary pump material 292 is in fluidic communication with a secondary pump material 294 having the same or lower fluid resistance than the primary material. The secondary material 294 may, in turn, be in fluidic communication with an optional tertiary pump material 296 having the same or lower fluid resistance than the secondary material. Additional adaptive components may be included in the adaptive pump 290 to provide further adaptive capacity for accumulating sweat, and thereby prevent excess sweat from backing up in the sweat path 210.

FIGS. 2A-2D depict device 200 having adaptive sweat flow components in the collector, channel and pump. However, in some embodiments, sweat sensing devices as described herein may include either an adaptive sweat collector, an adaptive channel, or an adaptive pump. In other embodiments, the sweat sensing device may include a combination of any two of an adaptive sweat collector, channel and pump. The selection of any one, two or three of an adaptive sweat collector, adaptive channel, and/or adaptive pump will be a design consideration based on the excessive sweat flow to be managed. Additionally, each of the adaptive channel, collector, or pump components described above may be configured with one, two, three, or more materials or layers that have the same or a progressively lower fluid resistance than the primary material. Additionally, while the different microfluidic materials have been depicted and described herein as being stacked relative to each other, the materials may be arranged in other configurations, such as, for example, in rows, side by side, or concentric, without departing from the purposes described herein. In alternative embodiments, a sweat sensing device, similar to that described above, may include an adaptive sweat collector, an adaptive channel, and/or an adaptive pump each having a primary material and one or more adaptive components, in which successive components have the same or a progressively increasing fluid resistance.

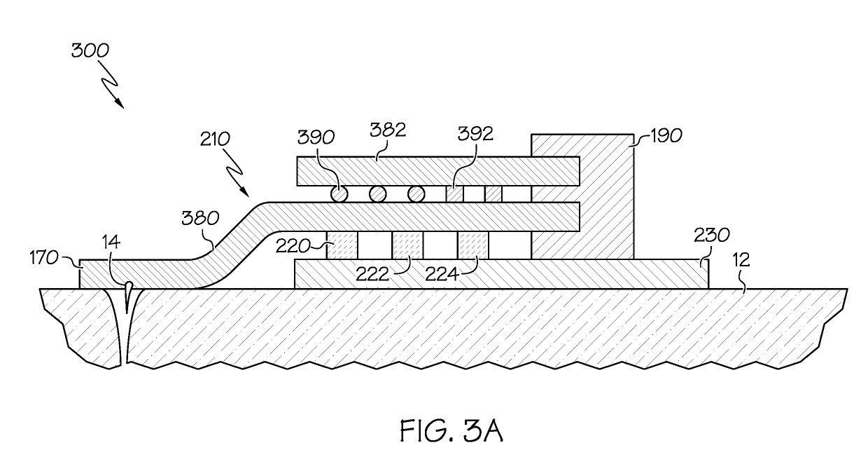

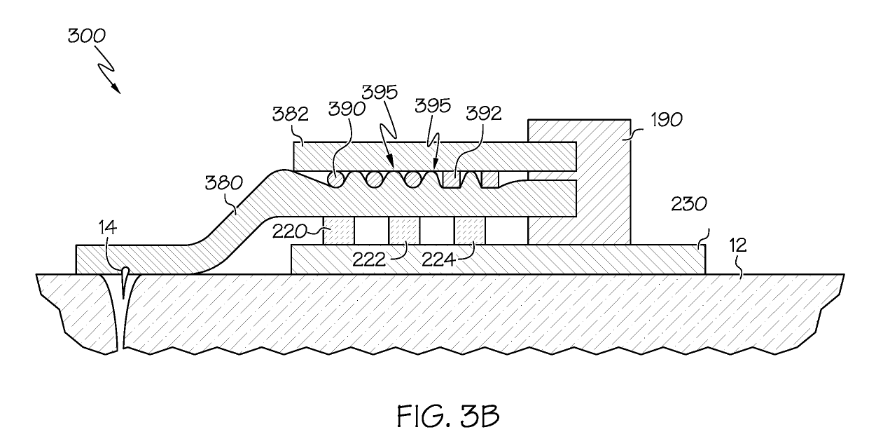

With reference to FIG. 3A, in another exemplary embodiment, a wearable sweat sensing device 300 includes one or more adaptive components, such as, for example, a collector, a channel, or a pump configured to allow the device to accommodate periods of high sweat rates. In this embodiment, the one or more sensors 220, 222, 224 are in fluidic communication with a primary channel material 380 that is also in fluid communication with skin 12. The device also includes a secondary channel material 382 having a fluid resistivity that is less than or equal to that of the primary material 380. The primary material 380 is separated from the secondary material 382 by a spacing component that includes a plurality of spherical spacers 390, or porous membrane materials 392. Exemplary porous membrane materials suitable for use herein can include track etched polycarbonate membranes of various pore sizes, pore densities, and hydrophobicity, or various types of woven nylon materials. At lower sweat generation rates, sweat will flow across the sensors 220-224 at a sweat sampling rate, and will partially fill the primary material 380, causing it to swell to a first thickness.

With reference to FIG. 3B, at higher sweat generation rates, the primary channel material 380 will swell to a second thickness and make contact with the secondary channel material 382, by crossing between the spacers 390, 392. When sweat contacts the secondary material 382, it will flow readily from the primary material to the secondary material at the contact junctions 395 that form. The spacers 390, 392 can be of a material that is hydrophobic and rigid, so that external deformations and saturation do not impact the spacers' ability to separate the primary and secondary materials during periods of low sweat rates. The thickness of the spacers is less than or equal to the thickness of the primary material at the first thickness, when it is saturated with sweat at the desired low sweat rate. While the exemplary embodiment of FIGS. 3A and 3B has been shown and described with respect to an adaptive channel between a collector 170 and pump 190, adaptive components may also be provided in the collector 170 and/or pump 190 in a similar fashion using spacers 390, 392 to separate the components. Furthermore, additional adaptive layers may be stacked above the primary and secondary materials 380, 382, and separated therefrom by additional spacers, in order to provide greater sweat capacity for the device.

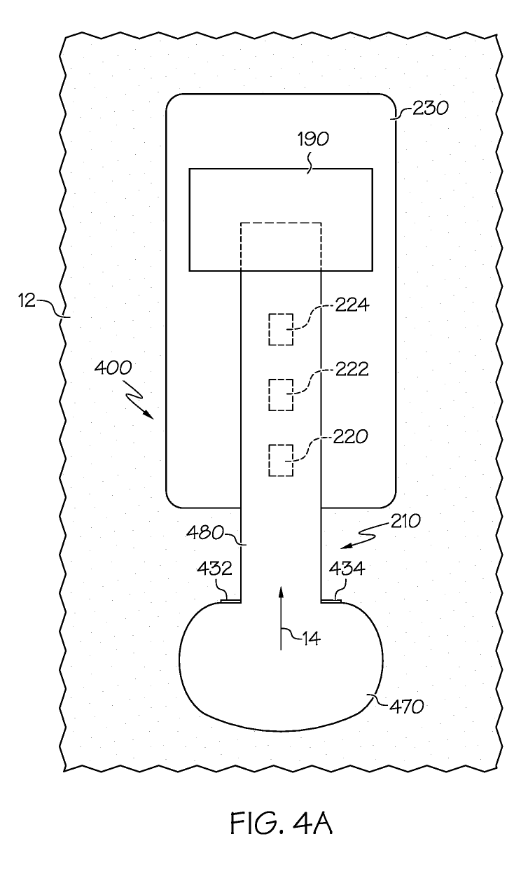

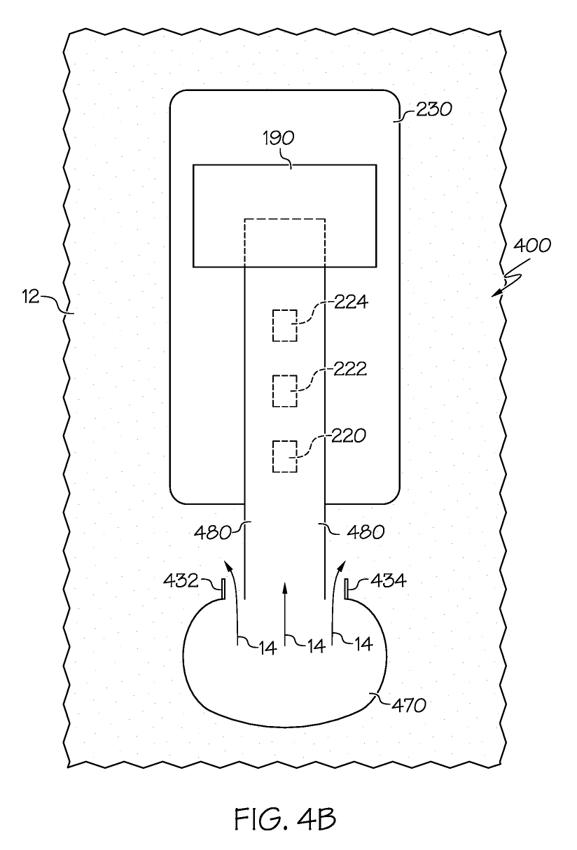

In another exemplary embodiment shown in FIGS. 4A and 4B, a sweat sensing device 400 may include an adaptive sweat collector 470 for delivering an adequate sweat sample to the device sensors 220-224 at low sweat rates, and removing excess sweat from the collection area at higher sweat rates. In this embodiment, the adaptive sweat collector 470 may include a plurality of check valves 432, 434 associated with the collector. As shown in FIG. 4A, at lower sweat rates, the check valves 432, 434 remain closed, and the sweat sample flows through the sweat path 210 from the collector 470 into the channel 480 across sensors 220-224. Referring to FIG. 4B, at higher sweat rates at least one check valve 432, 434 opens, allowing a portion of the excess sweat sample 14 to be diverted out through the valves prior to entering the channel 480. Once diverted, the sweat 14 may be moved to a microfluidic pump (not shown), or may simply evaporate off the device.

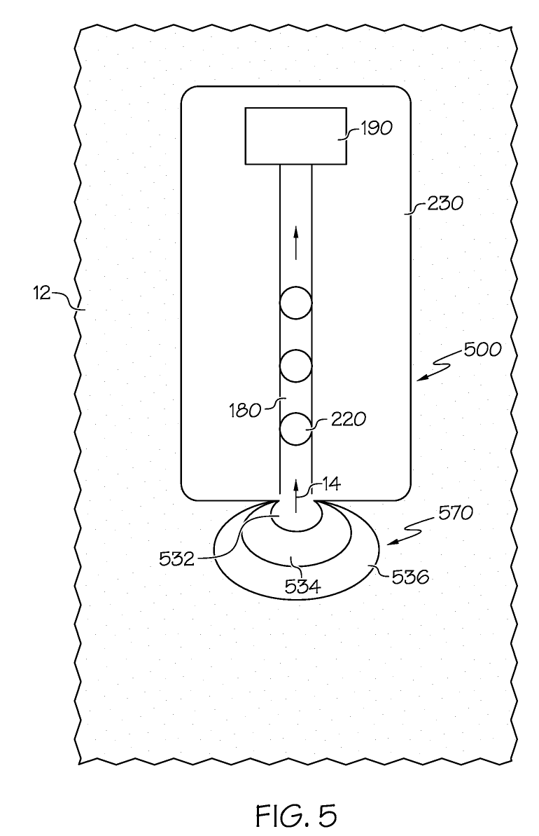

In another exemplary embodiment, depicted in FIG. 5, a sweat sensing device 500 includes an adaptive sweat collector 570 having a plurality of collection areas that activate or deactivate depending on the amount of sweat being generated. Such activation or deactivation may be, for example, through the use of materials with different wicking pressures or fluidic resistances. The adaptive sweat collector 570 may be configured in a concentric model as shown, or another suitable configuration. In such embodiments, the sweat collector 570 has a first section 532 closest to the sweat channel 180, that has a higher wicking pressure, and a second section 534 having a lower wicking pressure. Additional sections may also be added, as indicated at 536, with the same or different wicking pressures.

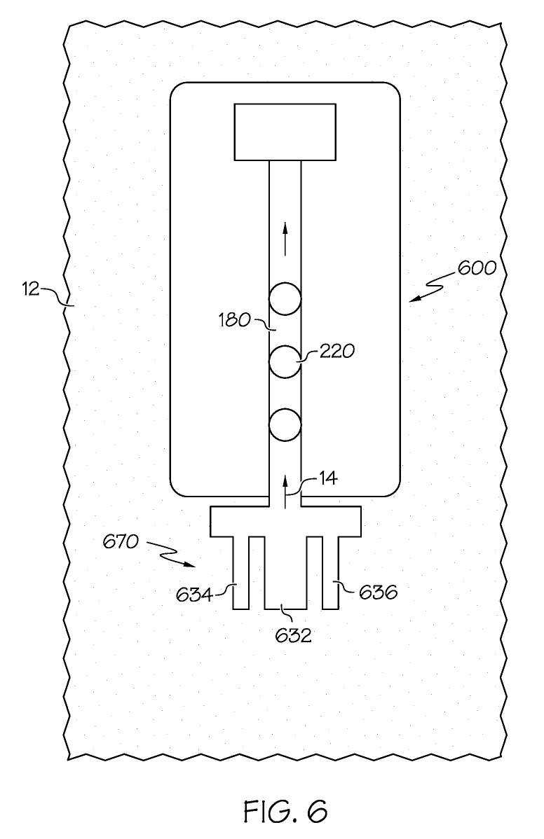

In another embodiment, shown in FIG. 6, a sweat sensing device 600 includes an adaptive sweat collector 670 arranged in a finger configuration. In this embodiment, a primary collector 632, has a first, higher wicking pressure, while a plurality of secondary collectors 634, and 636, have lower wicking pressures.

In addition to the above described device modifications, a sweat sensing device may also be configured to accommodate periods of high sweat rates (or even very low sweat rates) through algorithms executed by the device's computing capability. Algorithms may be programmed to account for the range of sweat generation rates at which the device operates, with the sweat concentration calculations differing based on the operating sweat rate range of the device. Such algorithms may, for example, filter data from sweat rates that are outside the device's operating range. For example, if a device were only capable of generating valid data when sweat rates are between 0.4 μμL/cm²/min and 1.0 μL/cm²/min, the algorithm would discard data from sweat rates of 1.5 μL/cm²/min. In other embodiments, the algorithm may weight data based on the measured sweat rate for the sweat sample on which the data is produced.

While several exemplary embodiments have been described for managing excess sweat flow, it is anticipated that other materials, elements and configurations may also be used, provided the alternative materials, elements and/or configurations provide chronological assurance and accurate measurements during periods of excessive sweat generation. Various modifications, alterations, and adaptations to the embodiments described herein may occur to persons skilled in the art with attainment of at least some of the advantages. The disclosed embodiments are therefore intended to include all such modifications, alterations, and adaptations without departing from the scope of the embodiments as set forth herein.

What is claimed is:

1. A wearable sweat sensing device capable of managing excess sweat flow, the device comprising:
at least one sweat sensor;
a sweat path for fluidly communicating a sweat sample from skin across the at least one sensor; and
at least one adaptive component associated with the sweat path for removing excess sweat from the sweat path.

2. The sweat sensing device of claim 1, wherein the adaptive component comprises at least one of: an adaptive collector; an adaptive channel; and an adaptive pump.

3. The sweat sensing device of claim 1, wherein the adaptive component comprises a primary material in fluidic communication with the sweat path and at least one additional material, and the additional material has an equal or lower fluid resistance than the primary material layer.

4. The sweat sensing device of claim 1, wherein the adaptive component comprises a primary material in fluidic communication with the sweat path and at least one additional material, and the additional material has an equal or higher fluid resistance than the primary material layer.

5. The sweat sensing device of claim 1, wherein the adaptive component comprises at least two materials for moving sweat relative to the sweat path, where the materials have one of the following:
different fluid resistances; different wicking pressures.

6. The sweat sensing device of claim 1, wherein the adaptive component comprises a wicking material.

7. The sweat sensing device of claim 1, wherein the adaptive component removes sweat from the sweat path to maintain a sweat sampling rate.

8. The sweat sensing device of claim 7, wherein the adaptive component moves sweat away from the sweat path to maintain a chronologically assured sweat sampling rate.

9. The sweat sensing device of claim 1, wherein the adaptive component comprises at least one check valve, where the valve functions to divert excess sweat from the sweat path.

10. The sweat sensing device of claim 9, wherein the at least one check valve is in fluidic communication with a sweat collector.

11. The sweat sensing device of claim 1, wherein the adaptive component comprises a primary material in fluid communication with a sweat path, a secondary material, and one or more spacers between the primary material and the secondary material.

12. A wearable sweat sensing device that provides excess sweat flow management, comprising:
at least one sensor specific to a sweat analyte; and
an adaptive component, the adaptive component comprising at least one of an adaptive pump, an adaptive channel, or an adaptive collector.

13. The wearable sweat sensing device of claim 12, wherein the adaptive component comprises at least two layers of wicking material having different fluid resistances for moving sweat relative to a sweat path.

14. The wearable sweat sensing device of claim 12, wherein the adaptive component comprises a primary wicking layer in fluid communication with a sweat path and at least one additional wicking layer in fluid communication with the primary wicking layer, the additional wicking layer having a fluid resistance equal to or less than the primary wicking layer.

15. The wearable sweat sensing device of claim 12, wherein the adaptive component comprises a primary wicking layer in fluid communication with a sweat path and at least one additional wicking layer in fluid communication with the primary wicking layer, the additional wicking layer having a fluid resistance equal to or greater than the primary wicking layer.

16. A method of managing excess sweat flow in a sweat sensing device configured to be worn on an individual's skin, comprising:
conveying a sweat sample from the skin into the device at a sweat flow rate;
conveying the sweat sample through a sweat path and across at least one sweat sensor, where the sweat path has a maximum flow capacity; and
using an adaptive component to convey excess sweat away from the sweat path when the sweat flow rate exceeds the maximum flow capacity.

17. The method of claim 16, wherein excess sweat is conveyed from the sweat path when the sweat flow rate causes the sweat sample to saturate the sweat path.

18. The method of claim 16, wherein the method further comprises managing excess sweat flow when the sweat path is tuned to operate at low sweat generation rates.

19. The method of claim 16, wherein the step of conveying excess sweat flow from the sweat path further comprises conveying sweat from the sweat path using layered adaptive components having equal or increasing fluid resistance.

20. The method of claim 16, wherein the step of conveying excess sweat flow from the sweat path further comprises conveying sweat from the sweat path using layered adaptive components having equal or decreasing fluid resistance.

21. The method of claim 16, wherein the step of conveying excess sweat flow from the sweat path further comprises using an adaptive component comprising at least one of the following: an adaptive collector, an adaptive channel, or an adaptive pump.

22. The method of claim 16, wherein the step of drawing excess sweat flow from the sweat path further comprises diverting sweat away from the sweat path using at least one check valve.
